(19) United States
(12) Patent Application Publication
(10) Pub. No.: US 2011/0008824 A1
(43) Pub. Date: Jan. 13, 2011

(54) DEVICE AND METHOD OF BACTERIA DETECTION

(75) Inventors: Jae Jin Lee, Daejeon (KR); Chung Hwan Kim, Daejeon (KR); Min Gun Kim, Daejeon (KR)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: TELECOMMUNICATION & ELECTRONICS INC., Daejeon (KR)

(21) Appl. No.: 12/520,253

(22) PCT Filed: Aug. 28, 2008
(86) PCT No.: PCT/KR08/05044

§ 371 Date: Sep. 29, 2010

(30) Foreign Application Priority Data
Jul. 8, 2008 (KR) 10-2008-0066233

(51) Int. Cl. C12Q 1/04; C12M 1/42

(52) U.S. Cl. 435/34; 435/288.7

(57) ABSTRACT

A bacteria measurement device and method are provided, which separate bacteria from a target object and attach the bacteria to an antibody with fluorescein isothiocyanate applied thereto, generate light using a light emitting unit, and excite the fluorescein isothiocyanate, and in which the excited fluorescein isothiocyanate secondarily emits light, and a light receiving unit receives a light output signal generated through the secondary emission, converts the light output signal into an electrical signal, and converts an analog signal which is the electrical signal into a digital signal, thus measuring the amount and type of bacteria.

### CROSS-REFERENCE TO RELATED APPLICATIONS

This application is the U.S. national phase of the International Patent Application No. PCT/KR2008/005044 filed Aug. 28, 2008, which claims the benefit of Korean Application No. 10-2008-0066233 filed Jul. 8, 2008, the entire content of which is incorporated herein by reference.

### BACKGROUND

The present invention relates, in general, to a bacteria measurement device and method. Various embodiments of the invention relate to a bacteria measurement device and method, which separate bacteria from a target object and attach the bacteria to an antibody with fluorescein isothiocyanate applied thereto, generate light using a light emitting unit, and excite the fluorescein isothiocyanate, and in which the excited fluorescein isothiocyanate secondarily emits light, and a light receiving unit receives a light output signal generated through the secondary emission, converts the light output signal into an electrical signal, and converts an analog signal which is the electrical signal into a digital signal, thus measuring the amount and type of bacteria.

Generally, since bacteria are not visible and cause various pathological phenomena, it is very important to sufficiently prevent bacteria. In order to measure the bacteria which are pathogens in real time, the bacteria need at a specific point in time to be proliferated and cultured in a uniform environment, so that the number of bacteria doubles, and thus the type and amount of bacteria may be detected through observation with a microscope.

Further, in the conventional technology, bacteria are gathered and are applied to a tool containing nutritive substance, so that an environment enabling bacteria to grow well is constructed, and, in that environment, bacteria are typically cultured for 24 to 48 hours, and thus the type and amount of bacteria present in the environment are measured using a microscope and a counter.

Such a bacteria measurement method is a very accurate and scientific method. However, there is a disadvantage in that, since the amount of bacteria must be measured after the bacteria has been cultured for 24 to 48 hours, a long time is required, thus making it inconvenient to use the method in places such as collective feeding facilities in which bacteria information must be detected in a short period of time.

### SUMMARY

The present invention has been made keeping in mind the above problems, and an object of the present invention is to provide a bacteria measurement device and method, which can measure bacteria by attaching bacteria to an antibody with fluorescein isothiocyanate (RTC) applied thereto so as to measure the amount of bacteria in a short period of time without needing to culture the bacteria.

In order to accomplish the above object, various embodiments of the present invention provide a bacteria measurement device, comprising a power supply unit for supplying voltage; a constant voltage circuit for maintaining the voltage supplied by the power supply unit at a constant voltage; a light emitting unit for emitting light to irradiate a sample, a light receiving unit for receiving light output signals from the sample and converting the light output signals into electrical signals when the sample is exposed to the light by the light emitting unit and then fluorescein isothiocyanate of the sample secondarily emits light; an Analog/Digital (A/D) converter for converting the electrical signals received through the light receiving unit into digital signals; a shift register for providing a measurement start signal required to measure the electrical signals and a signal required to receive measured values; a processor for sequentially managing an entire system and transmitting data obtained from the sample to a display; and the display for externally outputting the data.

The fluorescein isothiocyanate may be applied to a first end of an antibody and the bacteria are attached to a second end of the antibody.

Further, in order to accomplish the above object, various embodiments of the present invention provide a bacteria measurement method, comprising a first step of disposing a sample between a light emitting unit and a light receiving unit; a second step of the light emitting unit emitting light to irradiate the sample; a third step of fluorescein isothiocyanate of the sample secondarily emitting light in response to the emitted light; a fourth step of the light receiving unit converting light output signals from the sample into electrical signals; a fifth step of sequentially receiving measured signals and measured values of the electrical signals through a shift register, a sixth step of converting the electrical signals output from the light receiving unit into digital signals using an Analog/Digital (A/D) converter; and a seventh step of outputting data converted into the digital signals.

The sample at the first step may be collected by a process comprising the steps of putting an analysis target object in a first culture bottle, and separating the analysis target object from bacteria by shaking the first culture bottle for about 30 to 60 seconds using deionized water; providing a funnel and a filter on a top of a second culture bottle, separating the analysis target object and the bacteria, contained in the first culture bottle, from each other, and then putting the bacteria in the second culture bottle after; sucking up the analysis target object filtered by the second culture bottle using a first injector; injecting the analysis target object into a filter case equipped with a filter using the first injector which sucked up the analysis target object, thus catching the bacteria; injecting an antibody, to which fluorescein isothiocyanate is applied, into the filter case; injecting water into the filter case equipped with the filter, using a second injector filled with water, thus enabling fluorescein isothiocyanate, which is not linked to bacteria in the filter case, to flow out from the filter case; injecting air into the filter case using a third injector so as to externally discharge water and fluorescein isothiocyanate remaining in the filter case; and separating the filter which caught bacteria from the filter case, and using the filter as the sample.

The bacteria measurement device and method according to various embodiments of the present invention are advantageous because bacteria can be measured in real time before eating the food containing them, without requiring a procedure for culturing bacteria, in order to prevent the occurrence of contamination attributable to bacteria, thus preventing a disease from striking many people.

Further, various embodiments of the present invention are advantageous because the amount and type of bacteria can be measured onsite without requiring a procedure for proliferating and culturing bacteria, thus reducing the time required for bacteria measurement.

### BRIEF DESCRIPTION OF THE DRAWINGS

FIG. 1 is a block diagram showing a bacteria measurement device according to an embodiment of the present invention;

FIG. 2 is a diagram showing a sample used in a bacteria measurement device according to an embodiment of the present invention;

FIG. 3 is a diagram showing the step of extracting a sample used in a bacteria measurement device according to an embodiment of the present invention; and

FIG. 4 is a flowchart showing a bacteria measurement method according to an embodiment of the present invention.

### DETAILED DESCRIPTION OF THE EMBODIMENTS

Hereinafter, embodiments of the present invention will be described in detail with reference to the attached drawings.

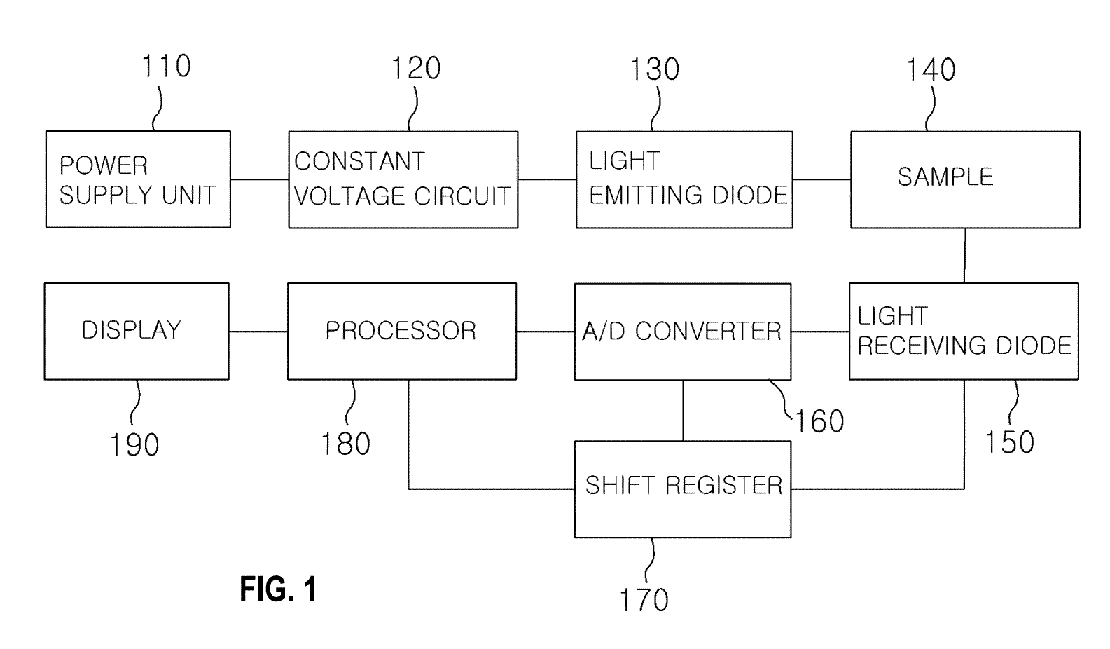

FIG. 1 is a diagram showing a bacteria measurement device according to an embodiment of the present invention.

As shown in FIG. 1, the bacteria measurement device includes a power supply unit 110, a constant voltage circuit 120, a light emitting unit 130, a light receiving unit 150, an Analog/Digital (A/D) converter 160, a shift register 170, a processor 180, and a display 190.

The power supply unit 110 supplies voltage to the entire bacteria measurement device.

The constant voltage circuit 120 maintains the voltage supplied by the power supply unit 110 at a constant voltage. The constant voltage circuit 120 of the present invention preferably maintains the voltage at 5V, but is not limited to any specific voltage.

When the light emitting unit 130 irradiates a sample 140, the processor 180 and the shift register 170 sequentially transmit a corresponding light signal to the light receiving unit 150. The light emitting unit 130 preferably has a wavelength of 490 nm or less. Further, the light emitting unit 130 is preferably implemented using a light emitting diode or a laser.

In this case, the light receiving unit 150 sequentially receives the light output signals of the sample, which are secondarily emitted by the sample 140, having received light from the light emitting unit 130, due to fluorescein isothiocyanate, and converts the light output signals into electrical signals, and thus transfers the electrical signals to the A/D converter 160. At this time, when the electrical signals, that is, the light emission wavelength and intensity of the fluorescein isothiocyanate, are measured, the type of bacteria and the relative amount of gathered bacteria can be detected. Here, the light receiving unit 150 may preferably control measurement in a 520 nm band. Further, the light receiving unit 150 may be preferably implemented as a photodiode or a photomultiplier tube.

The A/D converter 160 converts the electrical signals output from the light receiving unit 150 into digital signals and transmits the digital signals to the processor 180.

The shift register 170 provides a measurement start signal required to measure the electrical signals and a signal required to receive measured values.

The processor 180 sequentially manages the entire system and transmits the digital signals provided by the A/D converter 160 to the display 190.

The display 190 outputs data received from the processor 180. In this case, the display 190 of the present invention may preferably output the amount and type of bacteria, but the data to be output is not limited and additional information about bacteria such as the indication of the dangerousness of the bacteria may be output. The data may be digital signals obtained in such a way that the light output signals obtained due to the fluorescein isothiocyanate are converted into electrical signals by the light receiving unit and the electrical signals are converted into the digital signals, and such data is preferably data about the bacteria.

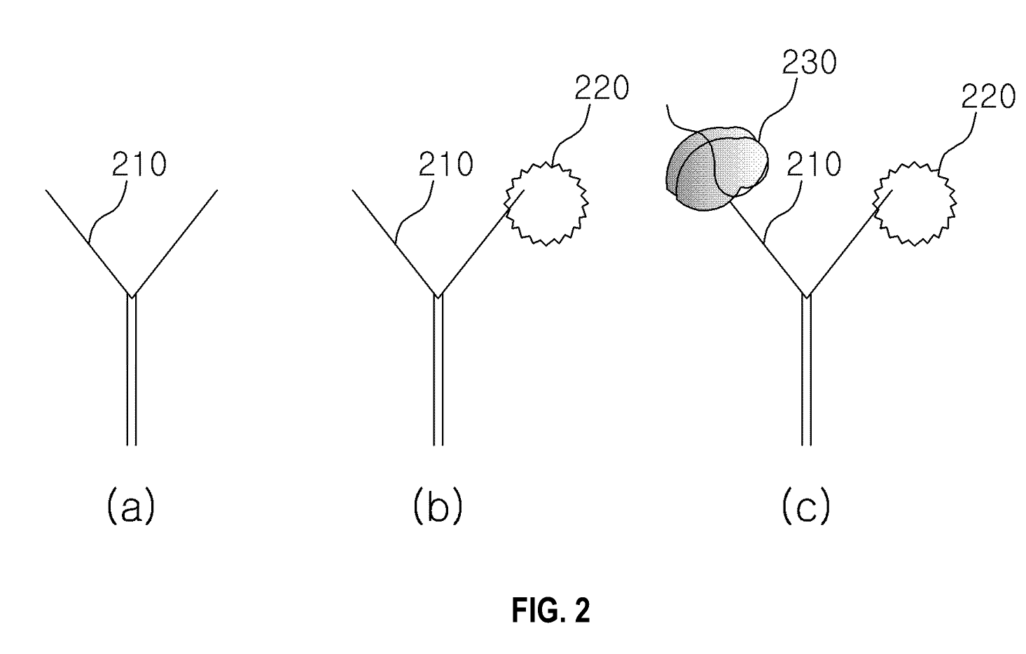

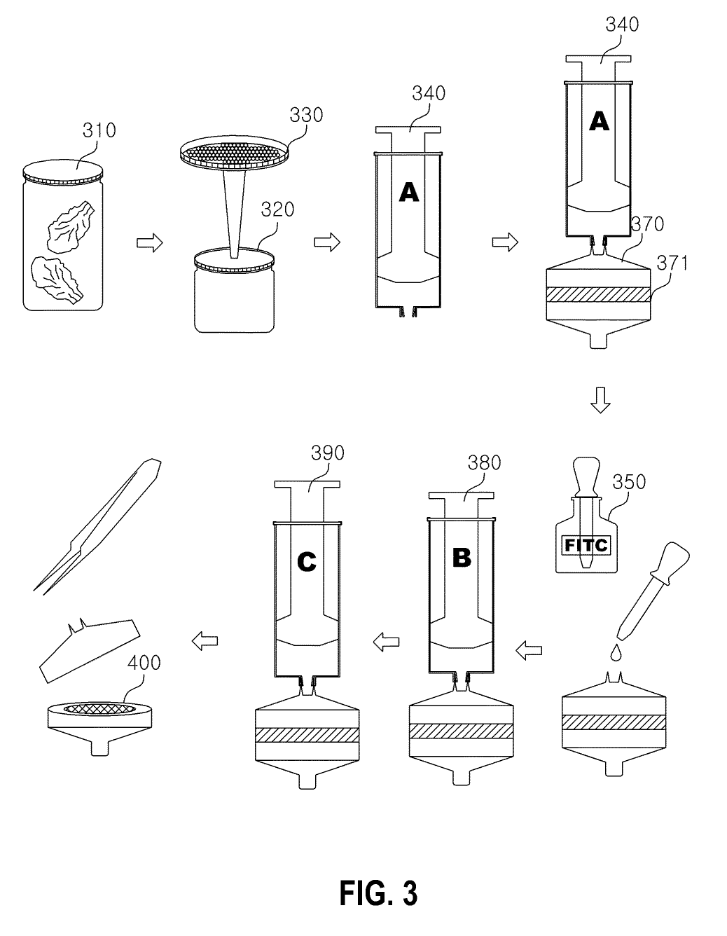

FIG. 2 is a diagram showing a sample used in the bacteria measurement device, and FIG. 3 is a diagram showing the step of extracting a sample used in the bacteria measurement device.

As shown in FIGS. 2 and 3, a sample 200 used in the bacteria measurement device according to the present invention denotes a structure in which a bacterium 230 is attached to an antibody 210 to which fluorescein isothiocyanate 220 is applied. FIG. 2(a) illustrates the antibody itself, (b) illustrates a structure in which the fluorescein isothiocyanate is applied to one end of the antibody, and (c) illustrates a structure in which the bacterium is attached to the other end of the antibody having one end to which the fluorescein isothiocyanate is applied.

The sample 200 of the present invention will be described in detail below.

The antibody 210 is a protein molecule having a function of eliminating foreign substances, is also called an immunoglobulin molecule, and is produced in such a way that a B cell reacts with an antigen when encountering the antigen. It is apparent that the antibody 210 is a protein mainly produced with the object of eliminating antigens, and that antibodies have peculiarities depending on antigens and specific antibodies have properties of acting only on specific antigens, and thus, if reacting antibodies can be known, antigens can also be known. For example, a salmonella antibody reacts only with a salmonella antigen, and a pathogenic antibody reacts only with a pathogenic antigen. In this way, in order to measure specific antigens, antibodies acting on the specific antigens are used, but, preferably, multiple antibodies may be mixed and used.

In this case, the antibody 210 is indicated in a ‘Y’ shape, in which the ends of respective three parts are portions to be individually linked. The fluorescein isothiocyanate (RTC) 220 is applied to one of the ends. Preferably, the maximum excitation wavelength of the RTC 220 is 490 nm and the maximum fluorescence wavelength is preferably 520 nm. Therefore, when the RTC 220 is applied to the antibody, and fluorescence emitted from they fluorescein isothiocyanate 220 through both the light emitting unit and the light receiving unit is measured, the same effect as that obtained when the antibody 210 is measured may be realized.

A method of collecting a sample according to the present invention will be described below.

First, an analysis target object is put in a first culture bottle 310, and the bottle 310 is shaken for about 30 to 60 seconds using deionized water, so that the analysis target object is separated from bacteria. At this time, the bacteria can spread into the deionized water and can be liquefied by inserting the analysis target object into the deionized water and shaking the first culture bottle 310.

Next, a funnel 300 is provided on the top of a second culture bottle 320, but a silk patch and filter paper are sequentially laid down inside the funnel 330, and the target object and the bacteria contained in the first culture bottle 310 are poured over the filter paper, and thus the bacteria are put in the second culture bottle 320.

Next, the analysis target object filtered by the second culture bottle 320 is sucked up using a first injector 340.

Next, the contents of the first injector 340 which sucked up the analysis target object are injected into a filter case 370 equipped with a filter 360, and thus the bacteria are caught by the filter. Here, “A” of the first injector 340 denotes the bacteria.

Next, the antibody with the fluorescein isothiocyanate 350 applied thereto is injected into the filter case 370. At this time, the bacteria contained in the filter case 370 are attached to the antibody with the fluorescein isothiocyanate 350 applied thereto.

Water is injected into the filter case 370 equipped with the filter 360 using a second injector 380 having water, so that the fluorescein isothiocyanate 350 not linked to the bacteria flows out of the filter case 370. At this time, the pore of the filter 360 preferably has a size sufficient to enable an antibody, which is linked only to the fluorescein isothiocyanate 350 without being linked to the bacteria, to be externally discharged. At this time, “B” of the second injector 380 denotes water.

Then, air is injected into the filter case 370 using a third injector 390 so as to externally discharge the water and fluorescein isothiocyanate 350 remaining in the filter case 370. At this time, “C” of the first injector 390 denotes air.

Finally, the filter 360 which caught the bacteria is separated from the filter case 370, and is used as a sample 400. Through the above procedure, the sample may be collected for a time period of about 10 to 20 minutes, thus measuring bacteria in a much shorter period than bacteria cultured for 24 to 48 hours in conventional technologies.

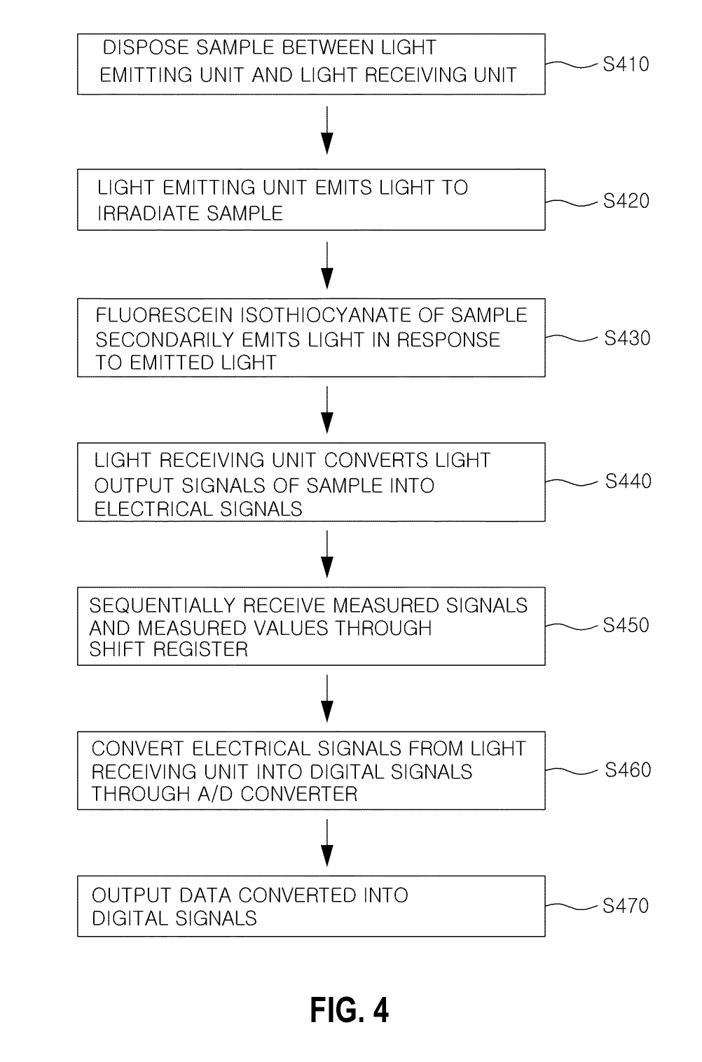

FIG. 4 is a flowchart showing a bacteria measurement method according to the present invention. A method of measuring a sample collected through the above method will be described below.

First, the sample is disposed between the light emitting unit and the light receiving unit at step S410. The sample is collected by the above-described sample collection method.

Then, the light emitting unit emits light to irradiate the sample at step S420.

Next, the fluorescein isothiocyanate of the sample secondarily emits light in response to the emitted light at step S430.

Next, the light receiving unit converts light output signals from the sample into electrical signals at step S440.

Next, measured signals and values of the electrical signals are sequentially received through the shift register at step S450.

The electrical signals from the light receiving unit are converted into digital signals by the A/D converter at step S460.

Finally, data converted into the digital signals is output at step S470.

Although the preferred embodiments of the present invention have been disclosed for illustrative purposes, those skilled in the art will appreciate that various modifications are possible, without departing from the scope and spirit of the invention. Therefore, the scope of the present invention should not be limited by the above embodiments, but should be defined by the accompanying claims and equivalents thereof.

Currently, in order to measure bacteria in real time, the bacteria need to be proliferated and cultured in a uniform environment, so that the number of bacteria doubles, and thus the type and amount of bacteria may be detected through observation with a microscope. Therefore, there is a disadvantage in that, since bacteria are typically cultured for 24 to 48 hours to measure the amount of bacteria, a long time is required, thus it is not easy to use the present invention in places used by a lot of people, such as collective feeding facilities for which information about bacteria must be detected in a short period of time. Therefore, a need for the present invention is predicted to increase from the standpoint of public benefit in collective feeding facilities and public restaurants for which the results of bacteria measurement must be rapidly acquired and which are used together by a lot of ordinary people.

### TABLE OF REFERENCE CHARACTERS

110 power supply unit
120 constant voltage circuit
130 light emitting unit
140, 400 sample
150 light receiving unit
160 A/D converter
170 shift register
180 processor
190 display
210 antibody
220, 350 fluorescein isothiocyanate
230 bacteria
310 first culture bottle
320 second culture bottle
330 funnel
340 first injector
360 filter
370 filter case
380 second injector
390 third injector

1. A bacteria measurement device, comprising:
a power supply unit for supplying voltage;
a constant voltage circuit for maintaining the voltage supplied by the power supply unit at a constant voltage;
a light emitting unit for emitting light to irradiate a sample;
a light receiving unit for receiving light output signals from the sample and converting the light output signals into electrical signals when the sample is exposed to the light by the light emitting unit and then fluorescein isothiocyanate of the sample secondarily emits light;
an analog-to-digital (A/D) converter for converting the electrical signals received through the light receiving unit into digital signals;
a shift register for providing a measurement start signal required to measure the electrical signals and a signal required to receive measured values;
a processor for sequentially managing an entire system and transmitting data obtained from the sample to a display; and
the display for externally outputting the data.

2. The bacteria measurement device according to claim 1, wherein the fluorescein isothiocyanate is applied to a first end of an antibody and the bacteria are attached to a second end of the antibody.

3. A bacteria measurement method, comprising:
a first step of disposing a sample between a light emitting unit and a light receiving unit;
a second step of the light emitting unit emitting light to irradiate the sample;
a third step of fluorescein isothiocyanate of the sample secondarily emitting light in response to the emitted light;
a fourth step of the light receiving unit converting light output signals from the sample into electrical signals;
a fifth step of sequentially receiving measured signals and measured values of the electrical signals through a shift register;
a sixth step of converting the electrical signals output from the light receiving unit into digital signals using an analog-to-digital (A/D) converter; and
a seventh step of outputting data converted into the digital signals.

4. The bacteria measurement method according to claim 3, wherein the sample at the first step is collected by a process comprising the steps of:
putting an analysis target object in a first culture bottle, and separating the analysis target object from bacteria by shaking the first culture bottle for about 30 to 60 seconds using deionized water;
providing a funnel and a filter on a top of a second culture bottle, separating the analysis target object and the bacteria, contained in the first culture bottle, from each other, and then putting the bacteria in the second culture bottle after;
sucking up the analysis target object filtered by the second culture bottle using a first injector;
injecting the analysis target object into a filter case equipped with a filter using the first injector which sucked up the analysis target object, thus catching the bacteria;
injecting an antibody, to which fluorescein isothiocyanate is applied, into the filter case;
injecting water into the filter case equipped with the filter, using a second injector filled with water, thus enabling fluorescein isothiocyanate, which is not linked to bacteria in the filter case, to flow out from the filter case;
injecting air into the filter case using a third injector so as to externally discharge water and fluorescein isothiocyanate remaining in the filter case; and
separating the filter which caught bacteria from the filter case, and using the filter as the sample.
